(19) United States
(12) Patent Application Publication
(10) Pub. No.: US 2016/0331371 A1
(43) Pub. Date: Nov. 17, 2016

(54) NEEDLE FOR SUTURE AND SUTURE ASSEMBLY INCLUDING THE SAME

(71) Applicants: Hyun Jun KIM, Seoul (KR); Young Jae KIM, Seoul (KR)

(72) Inventors: Hyun Jun KIM, Seoul (KR); Young Jae KIM, Seoul (KR)

(21) Appl. No.: 15/155,415

(22) Filed: May 16, 2016

(30) Foreign Application Priority Data
May 15, 2015 (KR) 10-2015-0068174

(51) Int. Cl. A61B 17/06; A61B 17/04

(52) U.S. Cl. CPC A61B 17/06004; A61B 17/0401; A61B 17/06166; A61B 2017/0404; A61B 2017/06176; A61B 2017/06009; A61B 2017/06019

(57) ABSTRACT

Disclosed herein is a needle for being connected to a suture, including: a body having a tip portion disposed at one end portion thereof and a rear end portion disposed at the other end portion thereof; and a suture connection part formed in a side surface portion of the body, wherein the suture connection part includes a reception path extended toward a lengthwise axial portion of the body and receiving a portion of the suture therein, and a closure member constituting a portion of the body and deformed by external force after a portion of the suture is received in the reception path, thereby closing a portion of the reception path.

### CROSS-REFERENCE TO RELATED APPLICATIONS

This application claims priority from and the benefit of Korean Patent Application No. 10-2015-0068174, filed on May 15, 2015, which is hereby incorporated by reference for all purposes as if fully set forth herein.

### BACKGROUND

1. Field

The present invention relates to a needle for a suture and a suture assembly including the same, and more particularly, to a medical needle having a structure in which it is easily coupled to a suture, and a suture assembly including the same.

2. Discussion of the Background

Generally, a suture used for a surgical operation is manufactured and sold in a state in which it is attached and fixed to one end of a needle.

A connection structure between the needle and the suture according to the related art will be described. A cylindrical hole having a predetermined depth in an axial direction of the needle is formed at one end of the needle to which the suture is to be attached, and one end of the suture is inserted into the cylindrical hole.

After one end of the suture is inserted into the cylindrical hole, the cylindrical hole is filled with an adhesive material to fix the suture to the cylindrical hole of the needle.

However, in the connection structure between the needle and the suture according to the related art, a process of connecting the suture to the needle may not be simply executed by a medical team in an operating room.

As a method for solving the problem described above, according to another related art, the suture is fixed to the cylindrical hole of the needle by a method of inserting the suture into the cylindrical hole of the needle and then applying pressure to a distal end of the needle to change a cross section of the cylindrical hole.

However, also in this structure, it is not easy to fit an end portion of the suture into the cylindrical hole of an end portion of the needle in the case of considering a size of the suture, such that it is not easy to connect and couple the suture to the needle by the medical team, and it is also not easy to adjust a length of the suture. Therefore, the suture is appropriate for being manufactured as a ready-made article in a factory, but it is difficult to cut the suture by a required length and connect the suture to the needle by the medical team in the operating room.

### RELATED ART DOCUMENT

### Patent Document

(Patent Document 1) Korean Utility Model Registration No. 20-0343084

### BRIEF SUMMARY OF THE INVENTION

Exemplary embodiments of the present invention are to solve the problem according to the related art described above, and provide a needle for being connected to a suture capable of easily connecting the suture to the needle to fix the suture to the needle so as not to be separated from the needle and selecting a length of the suture as needed by a medical team and immediately connecting and coupling the suture having the selected length to the needle, and a suture assembly including the same.

According to an aspect of the present invention, a needle for being connected to a suture includes: a body having a tip portion disposed at one end portion thereof and being sharp and a rear end portion disposed at the other end portion thereof; and a suture connection part formed in a side surface portion of the body, wherein the suture connection part includes a reception path extended toward a lengthwise axial portion of the body and receiving a portion of the suture therein, and a closure member constituting a portion of the body and deformed by external force after a portion of the suture is received in the reception path, thereby closing a portion of the reception path (i.e. blocking a portion of the reception path opened to the outside).

The suture connection part may include a closure member contact end portion formed at an end portion of the closure member in order to close the reception path, and a contact surface formed near the side surface of the body and corresponding to the closure member contact end portion, such that the contact surface can come into contact with the closure member contact end portion when the closure member is deformed in order to close the reception path.

In a state in which the closure member contact end portion contacts the contact surface after the closure member closes the reception path, the closure member may be in a continuous surface state on a surface of the body.

The closure member may be formed on an upper surface of the body, and a length of the upper surface extended from the rear end portion to the tip portion may be shorter than that of a lower surface extended from the rear end portion to the tip portion.

Alternatively, the closure member may be formed on an upper surface of the body, and a length of the upper surface extended from the rear end portion to the tip portion may be longer than that of a lower surface extended from the rear end portion to the tip portion.

The reception path may be extended from an outer surface of the body to a lengthwise axial portion of the body, and a distance from the central portion of the body to the outer surface of the body in the radial direction may be a half of a diameter of the rear end portion.

In a needle according to another exemplary embodiment of the present invention, the suture connection part may include closure member contact end portions each formed at end portions of a pair of closure members so as to face each other in a circumferential direction in order to close a portion of the reception path, and a rear end opening hole that is in communication with the reception path and formed to penetrate through the rear end portion.

In a state in which a pair of closure member contact end portions contact each other after the closure members close a portion of the reception path, outer surfaces of the closure members may be in a continuous surface state on an outer surface of the body.

According to another aspect of the present invention, a suture assembly includes: the needle described above; and the suture penetrating through the reception path of the needle and then connected to the needle.

The suture may further include barbs which are extended from a surface of the suture, such that they are inclined away from the needle.

The suture assembly may further include a suture supporter connected to a remote part of the suture from the needle and having the suture penetrating therethrough, wherein the suture supporter is formed in a shape of a truncated cone, and a bottom surface having a larger diameter in the truncated cone is disposed toward the needle.

The suture assembly may further include a knot disposed adjacently to a surface having a smaller diameter in the suture supporter formed in the shape of the truncated cone in order to prevent the suture supporter from being separated from the suture.

The reception path may be extended in the radial direction of the body while at the same time being extended as a non-linear path toward the rear end portion.

### BRIEF DESCRIPTION OF THE DRAWINGS

FIG. 1 is a perspective view illustrating a needle of which a suture connection part is in an opened state according to an exemplary embodiment of the present invention.

FIG. 2 is a front view of the needle of FIG. 1.

FIG. 3 is a front view illustrating the needle of which the suture connection part is in a closed state forming connection between the needle of FIG. 1 and a suture.

FIG. 4 is a front view illustrating a suture assembly having a suture connected to the needle in a connection state of FIG. 3.

FIG. 5 is a perspective view illustrating a needle of which a suture connection part is in an opened state according to another exemplary embodiment of the present invention.

FIG. 6 is a perspective view illustrating a needle of which a suture connection part is in an opened state according to still another exemplary embodiment of the present invention.

FIG. 7 is a perspective view illustrating the needle of which the suture connection part is in a closed state forming connection between the needle of FIG. 6 and a suture.

FIG. 8 is a front view illustrating a suture assembly having a suture connected to the needle in a connection state of FIG. 7.

### DETAILED DESCRIPTION OF THE ILLUSTRATED EMBODIMENTS

The present invention will become apparent from exemplary embodiments to be described below in detail together with the accompanying drawings. However, the present invention is not limited to exemplary embodiments to be described below, but may be implemented in various different forms, these exemplary embodiments will be provided only in order to make the present invention complete and allow those skilled in the art to completely recognize the scope of the present invention, and the present invention will be defined by the scope of the claims. Meanwhile, terms used in the present specification are for explaining exemplary embodiments rather than limiting the present invention.

In the present disclosure, a singular form includes a plural form unless explicitly described to the contrary. Components, steps, operations, and/or elements mentioned by terms “comprise” and/or “comprising” used in the present disclosure do not exclude the existence or addition of one or more other components, steps, operations, and/or elements. Terms ‘upper portion’, ‘lower portion’, and the like, may be used to explain various components with reference to the accompanying drawings, but these components are not to be construed as being limited by these terms. The terms are used to distinguish one component from another component.

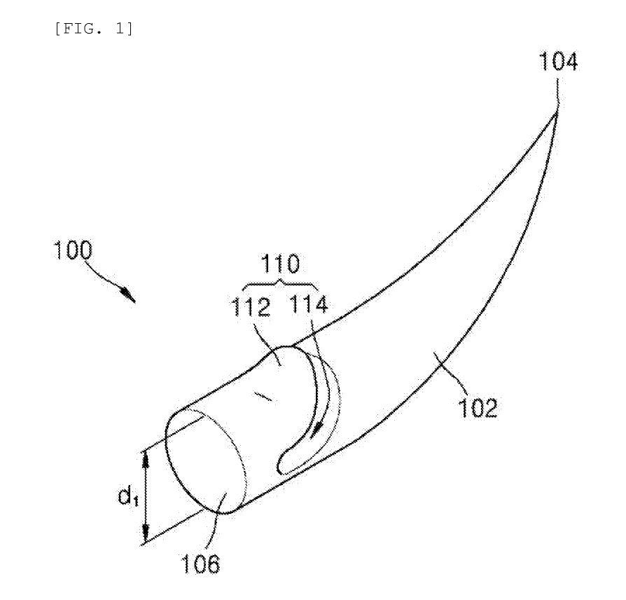

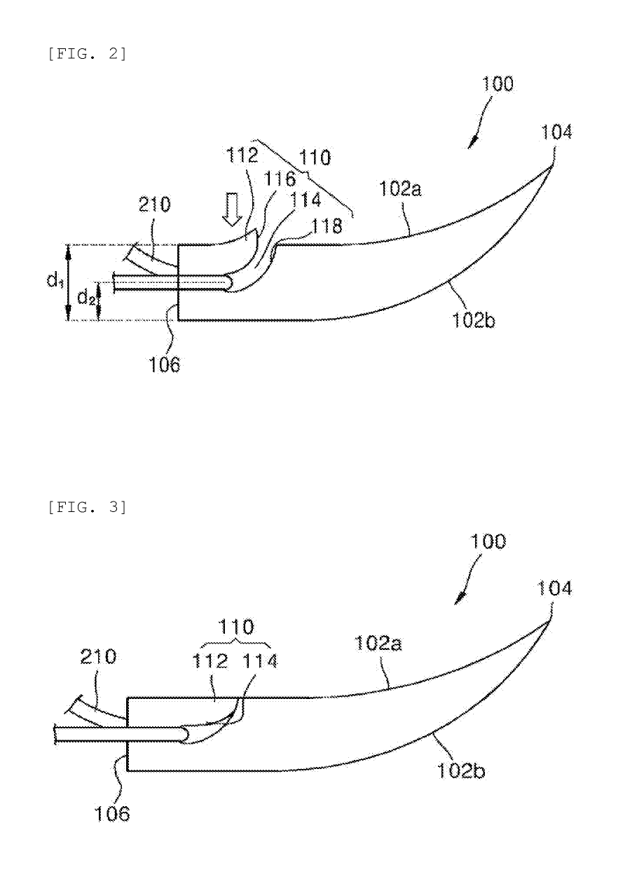

FIG. 1 is a perspective view illustrating a needle of which a suture connection part is in an opened state according to an exemplary embodiment of the present invention, and FIG. 2 is a front view of the needle of FIG. 1.

Referring to FIG. 1, the needle 100 according to an exemplary embodiment of the present invention includes a body 102 having a sharp tip portion 104 disposed at one end portion thereof and a rear end portion 106 disposed at the other end portion thereof and having a substantially circular cross section. The needle 100 is a needle for being connected to a suture.

The needle 100 includes a suture connection part 110 formed on a side surface of the middle of the body 102 extended in a length direction. The suture connection part 110 includes a reception path 114 which is extended toward a lengthwise axial portion of the body 102, and formed so as to receive a portion of the suture therein. In addition, the suture connection part 110 includes a closure member 112 for closing an inlet portion of the reception path 114.

The closure member 112 constitutes a portion of the body 102, and is deformed by external force (denoted by an arrow in FIG. 2) after a portion of the suture 210 is received in the reception path 114, thereby closing a portion of the reception path so that the suture is not moved through the portion of the reception body. The external force may be applied by a portable press, pliers, or the like.

The reception path 114 is extended as a non-linear path, which may be a curved path or a path bent at least once. Particularly, the reception path 114 is extended in a non-linear shape toward the rear end portion 106 when being extended from an inlet to the center of the body 102.

The suture connection part 110 includes a closure member contact end portion 116 formed at an end portion of the closure member 112 in order to close the reception path 114, and a contact surface 118 formed near the side surface of the body 102 and corresponding to the closure member contact end portion 116, such that it can come into contact with the closure member contact end portion 116 when the closure member is deformed in order to close the reception path 114.

FIG. 3 is a front view illustrating the needle of which the suture connection part is in a closed state forming connection between the needle of FIG. 1 and a suture.

Referring to FIG. 3, in a state in which the closure member contact end portion 116 contacts the contact surface 118 after the closure member 112 closes the reception path 114, the closure member 112 may be flush with an outer surface (or the side surface) of the body 102 or may be in a continuous surface state on an outer surface (or the side surface) of the body 102 without being protruded. Therefore, after the suture connection part 110 is closed, the outer surface of the body 102 of the needle 100 can be maintained in a smooth state, and thus, interference between the outer surface of the body 102 and a surrounding tissue can be avoided during suturing tissues.

As illustrated in FIGS. 1 to 3, in the needle 100 according to an exemplary embodiment of the present invention, the closure member 112 is formed on an upper surface 102a of the side outer surface of the body 102 extended in the length direction, and the body 102 may have a curved portion formed, for example, near the tip portion 104. That is, a length of the upper surface 102a extended from the rear end portion 106 to the tip portion 104 may be shorter than that of a lower surface 102b extended from the rear end portion 106 to the tip portion 104.

Therefore, in the needle 100 according to an exemplary embodiment of FIGS. 1 to 3, the suture connection part 110 is formed in a curved inner side portion of the curved needle 100.

Meanwhile, referring to FIG. 2, the reception path 114 is extended from the outer upper surface 102a of the body 102 toward a lengthwise axial portion of the body 102. Therefore, in the case in which a diameter of the rear end portion is d₁ on the basis of the rear end portion 106, a distance d₂ from the inner end of the reception path 114 to an outer surface (or the side surface) of the body in the radial direction can be set to be a half of the diameter of the rear end portion.

Therefore, when the suture 210 is received in the reception path 114, force that the suture 210 applies to the inner end portion of the reception path 114 of the body 102 by tension of the suture 210 may not cause a torque resulting from eccentricity of the force application point to act on a cross section of a portion near the rear end 106 of the body 102 in the radial direction while acting in equilibrium on the body 102 or may decrease such torque.

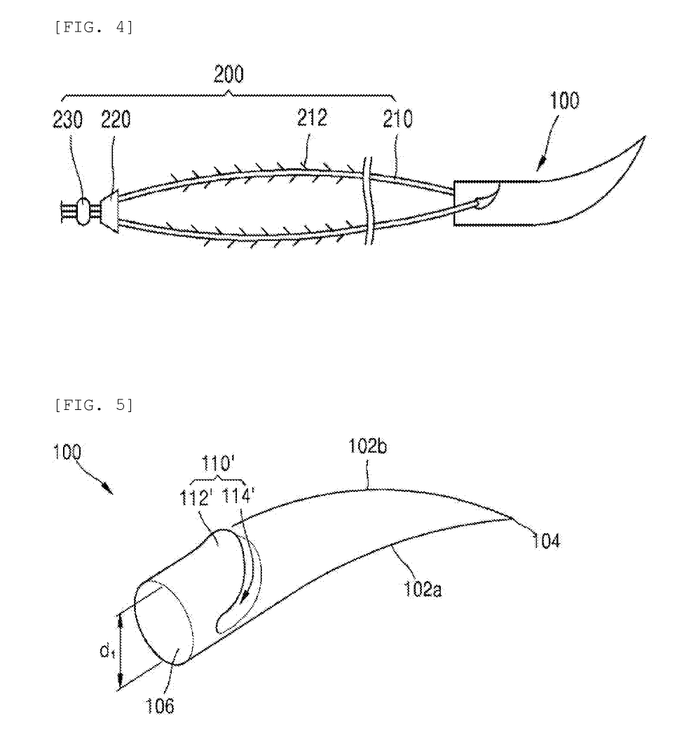

FIG. 4 is a front view illustrating a suture assembly having a suture connected to the needle in a connection state as illustrated in FIG. 3. In FIG. 4, sizes of the suture and the needle are exaggerated for convenience of explanation.

The suture assembly 200 illustrated in FIG. 4 may include the needle 100 and the suture 210 connected to the needle 100.

As described above with reference to FIGS. 1 to 3, the needle 100 according to an exemplary embodiment of the present invention includes the body 102 having the sharp tip portion 104 disposed at one end portion thereof and the rear end portion 106 disposed at the other end portion thereof and having a substantially circular cross section.

The needle 100 includes the suture connection part 110 formed on the side surface portion of the body 102 extended in the length direction. The suture connection part 110 includes the reception path 114 extended toward the lengthwise axial portion of the body 102 and formed so as to receive a portion of the suture therein. In addition, the suture connection part 110 includes the closure member 112 for closing the inlet portion of the reception path 114.

In the state in which the closure member contact end portion 116 contacts the contact surface 118 after the closure member 112 closes the reception path 114, the closure member 112 is flush with the outer surface of the body 102 without being protruded. Therefore, after the suture connection part 110 is closed, the outer surface of the body 102 of the needle 100 is maintained in the smooth state, and thus, the interference between the outer surface of the body 102 and the surrounding tissue cannot be caused during suturing the tissues.

Since the inlet of the reception path 114 is closed by the closure member 112 after a portion of the suture 210 is inserted in the reception path 114, the suture 210 can be connected to the needle 100, such that it penetrates through the reception path 114 of the needle 100.

Meanwhile, the suture 210 further includes a plurality of barbs 212 extended from the surface of the suture, such that they are inclined away from the needle 100. The suture is generally configured of one thread, but it is folded into two strands at the reception path 114 of the needle 100, such that the two strands of the suture 210 are extended as illustrated in FIG. 4, and two set of barbs formed on the two strands are oriented in opposite directions to each other with respect to the suture itself.

In addition, the suture assembly 200 further includes a suture supporter 220 connected to a remote part of the suture 210 from the needle and having the suture 210 penetrating therethrough. The suture supporter 220 is formed in a shape of a truncated cone. In this case, a bottom surface (a right side surface of the suture supporter in FIG. 4) having a larger diameter in the truncated cone is disposed toward the needle 100, such that the suture 210 can perform a anchoring function when it is placed within tissues.

Although not illustrated in the accompanying drawings, a plurality of cut parts may be formed on a lower portion of the truncated cone having the larger diameter in the suture supporter 220. In addition, the respective strands of the suture 210 folded at the reception path 114 of the needle 100 pass through a through-hole formed in the central portion of the suture supporter 220.

In addition, the suture assembly 200 further includes a knot 230 disposed adjacently to a surface (a left side surface of the suture supporter in FIG. 4) having a smaller diameter in the suture supporter formed in the shape of the truncated cone in order to prevent the suture supporter 220 from being separated from the suture 210. The knot 230 may be formed knotting the suture 210. However, the knot can be replaced by other disengagement prevention member (not shown). For example, each of two strands of the suture 210 may have a knot at their end portions.

The knot 230 needs to be formed to have an outer diameter larger than the through-hole formed in the suture supporter 220 in order to prevent the suture supporter 220 from being separated from the suture supporter 210.

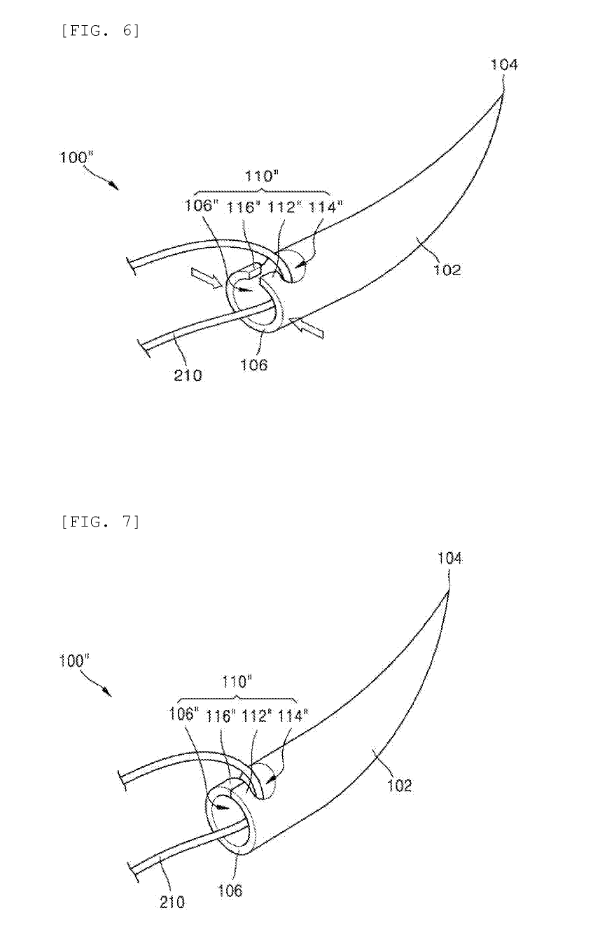

FIG. 5 is a perspective view illustrating a needle of which a suture connection part is in an opened state according to another exemplary embodiment of the present invention.

The needle illustrated in FIG. 5 is substantially the same as the needle according to an exemplary embodiment of FIGS. 1 to 3 except for a direction of curve of the needle.

Basically, in relation to a shape of the body 102 of the needle 100, a length of the upper surface (denoted by 102b in FIG. 5) extended from the rear end portion 106 to the tip portion 104 is longer than that of the lower surface (denoted by 102a in FIG. 5) extended from the rear end portion 106 to the tip portion 104. That is, the needle 100 has a shape in which it is curved downward in FIG. 5. In the shape of the body 102 of the needle 100 described above, the suture connection part 110′ is formed on the upper surface (denoted by 102b in FIG. 5) of the body, and the reception path 114′ is extended in a non-linear shape from the upper surface of the body toward the lengthwise axial portion of the body in the radial direction.

In addition, the closure member 112′ is also formed on the upper surface of the body.

Therefore, the suture connection part 110′ is formed on a curved outer surface of the needle.

Similar to an exemplary embodiment illustrated in FIGS. 1 to 3, when the closure member 112 is deformed by external force after the suture is received in the reception path 114′, the inlet of the reception path 114′ is closed and, the suture connection part 110′ can allow the upper surface of the needle 100 to be smoothly maintained to prevent the interference between the outer surface of the needle and the tissue at the time of inserting the needle into the tissue.

A position of the suture connection part according to an exemplary embodiment of the present invention may be variously changed. Although the needle is curved upward and the suture connection part is disposed on the upper surface of the body in an exemplary embodiment of FIG. 1, the needle may also be curved leftward or rightward rather than upward.

Although the needle is curved downward and the suture connection part is disposed on the upper surface of the body in an exemplary embodiment of FIG. 5, the needle may also be curved leftward or rightward rather than upward.

Optionally, in the needles according to exemplary embodiments of FIGS. 1 to 5, a round part or round indentation (not shown) may be formed in the needle at a portion at which the suture and the needle are connected to and contact each other in order to prevent abrasion between the suture and the needle.

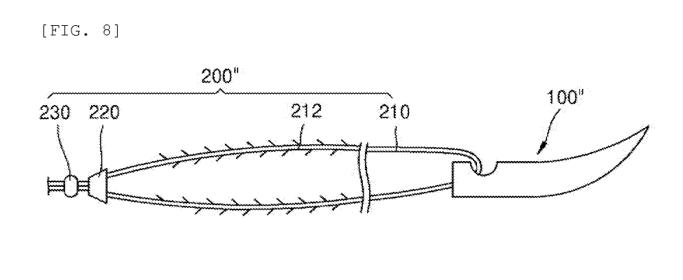

FIG. 6 is a perspective view illustrating a needle of which a suture connection part is in an opened state according to still another exemplary embodiment of the present invention, FIG. 7 is a front view illustrating the needle of which the suture connection part is in a closed state with the needle of FIG. 6 connected to a suture, and FIG. 8 is a front view illustrating a suture assembly having a suture connected to the needle in a connection state of FIG. 7.

The needle according to an exemplary embodiment illustrated in FIGS. 6 to 8, which is a needle 100″ having a tip portion 104 disposed at one end portion thereof and a rear end portion 106 disposed at the other end portion thereof and being to be connected to the suture, is similar to the needle according to an exemplary embodiment illustrated in FIGS. 1 to 4 in that it includes a suture connection part 110″ formed on a side surface of a body.

However, in a detailed configuration of the suture connection part, the suture connection part 110″ of the needle 100″ illustrated in FIGS. 6 to 8 includes closure member contact end portions 116″ separated from each other so as to face each other in a circumferential direction for closing a portion of the reception path 114″ formed in an inner diameter direction of the body and each formed at end portions of a pair of closure members so as to be deformed by external force to contact each other, and a rear end opening hole 106″ that is in communication with the reception path 114″ and formed to penetrate through the is rear end portion.

When the closure member contact end portions 116″ are spaced apart from each other, the reception path 114″ and the rear end opening hole 106″ are in communication with each other. Here, a space between the closure member contact end portions 116″ facing each other is in communication with the reception path 114″ and the rear end opening hole 106″. Here, the suture 210 is inserted into the space.

Then, when the pair of closure members 112″ facing each other are deformed by the external force (denoted by an arrow in FIG. 6) to contact each other, the space between the closure member contact end portions 116″ is removed, such that the suture penetrates through the reception path 114″ and the rear end opening hole 106″ and is then connected to the needle 100″.

Meanwhile, as illustrated in FIG. 7, in a state in which a pair of closure member contact end portions 116″ contact each other after the pair of closure members 112″ facing each other in the circumferential direction are deformed to contact each other, thereby closing a portion of the reception path 114″ (i.e. blocking a portion of the reception path opened to the outside), outer surfaces of the closure members are in a smoothly continuous surface state on an outer surface of the body 102, such that the needle is not unnecessarily caught by the tissue when the needle is moved within the tissue.

FIG. 8 is a front view illustrating a suture assembly having a suture connected to the needle in a connection state of FIG. 7. Similar to the form illustrated in FIG. 4, the needle 100″ is connected to the suture 210 to configure the suture assembly 200″.

A method of connecting the needle according to an exemplary embodiment of the present invention to the suture will be described. As illustrated in FIGS. 2 and 6, a worker (generally, a medical team) disposes the suture having a predetermined length in the opened reception path of the needle in a state in which the closure member of the needle is opened.

The closure member of the needle is pushed using a pressing tool in a state in which the suture is disposed in the reception path of the needle (arrows of FIGS. 2 and 6 denote external force applied by the pressing tool). Then, the reception path is closed in a state in which the suture is caught in the reception path of the needle by the closure member of the needle.

In the suture and the suture assembly according to the present invention, the following effects may be implemented.

First, the suture may be easily connected to the needle to thereby be fixed to the needle so as not to be separated from the needle.

Second, the medical team may select a length of the suture as needed, and immediately connect and couple the suture having the selected length to the needle.

Third, since the closure member is in the continuously smooth surface state on the surface of the body in the state in which the closure member contact end portion contacts the contact surface after the closure member closes the reception path, the needle may smoothly enter the tissue without the closure member of the needle being caught by the tissue when the needle enters the tissue from the tip portion.

Fourth, the reception path is extended up to the lengthwise axial portion of the body of the needle, and even tension acts on the suture of which a portion is received in the reception path, the suture allows force to act on the lengthwise axial portion of the needle, thereby preventing eccentric force from being applied to the needle.

Although the present invention has been described in relation to the above-mentioned exemplary embodiments, various modifications and alterations may be made without departing from the spirit and scope of the present invention. Therefore, these modifications and alterations fall within the scope of the claims as long as they belong to the scope of the present invention.

What is claimed is:

1. A needle for being connected to a suture, comprising:
a body having a tip portion disposed at one end portion thereof and a rear end portion disposed at the other end portion thereof; and
a suture connection part formed in a side surface portion of the body,
wherein the suture connection part includes a reception path extended toward a lengthwise axial portion of the body and receiving a portion of the suture therein, and a closure member constituting a portion of the body and deformed by external force after a portion of the suture is received in the reception path, thereby closing a portion of the reception path.

2. The needle of claim 1, wherein the suture connection part includes a closure member contact end portion formed at an end portion of the closure member in order to close the reception path, and a contact surface formed near the side surface of the body and corresponding to the closure member contact end portion, such that the contact surface can come into contact with the closure member contact end portion when the closure member is deformed in order to close the reception path.

3. The needle of claim 2, wherein in a state in which the closure member contact end portion contacts the contact surface after the closure member closes the reception path, the closure member is in a continuous surface state on an outer surface of the body.

4. The needle of claim 1, wherein the closure member is formed on an upper surface of the body, and a length of the upper surface extended from the rear end portion to the tip portion is shorter than that of a lower surface extended from the rear end portion to the tip portion.

5. The needle of claim 1, wherein the closure member is formed on an upper surface of the body, and a length of the upper surface extended from the rear end portion to the tip portion is longer than that of a lower surface extended from the rear end portion to the tip portion.

6. The needle of claim 1, wherein the reception path is extended from an outer surface of the body to a lengthwise axial portion of the body, and a distance from the central portion of the body to the outer surface of the body in the radial direction is a half of a diameter of the rear end portion.

7. The needle of claim 1, wherein the suture connection part includes closure member contact end portions each formed at end portions of a pair of closure members so as to face each other in a circumferential direction in order to close a portion of the reception path, and a rear end opening hole that is in communication with the reception path and formed to penetrate through the rear end portion.

8. The needle of claim 7, wherein in a state in which a pair of closure member contact end portions contact each other after the closure members close a portion of the reception path, outer surfaces of the closure members are in a continuous surface state on an outer surface of the body.

9. A suture assembly comprising:
the needle of claim 1; and
the suture penetrating through the reception path of the needle and then connected to the needle.

10. The suture assembly of claim 9, wherein the suture further includes barbs which are extended from a surface of the suture, such that they are inclined away from the needle.

11. The suture assembly of claim 9, further comprising a suture supporter connected to a remote part of the suture from the needle and having the suture penetrating therethrough,
wherein the suture supporter is formed in a shape of a truncated cone, and a bottom surface having a larger diameter in the truncated cone is disposed toward the needle.

12. The suture assembly of claim 11, further comprising a knot disposed adjacently to a surface having a smaller diameter in the suture supporter formed in the shape of the truncated cone in order to prevent the suture supporter from being separated from the suture.

13. A suture assembly comprising:
the needle of claim 7; and
the suture penetrating through the reception path of the needle and then connected to the needle.
